(19) United States
(12) Patent Application Publication
(10) Pub. No.: US 2024/0315834 A1
(43) Pub. Date: Sep. 26, 2024

(54) CROSSLINKED POLYMER COATINGS FOR INTRAOCULAR LENS (IOL) CARTRIDGES AND METHOD FOR CREATING THIS COATING ON THE INNER SURFACE OF THE CARTRIDGE

(71) Applicant: VSY BIYOTEKNOLOJI VE ILAC SANAYI ANONIM SIRKETI, ISTANBUL (TR)

(72) Inventors: Busra Gizem KAYA, ISTANBUL (TR); Faruk OYTUN, ISTANBUL (TR)

(73) Assignee: VSY BIYOTEKNOLOJI VE ILAC SANAYI ANONIM SIRKETI, ISTANBUL (TR)

(21) Appl. No.: 18/269,765

(22) PCT Filed: Dec. 28, 2021
(86) PCT No.: PCT/TR2021/051564

§ 371 Date: Jun. 27, 2023

(30) Foreign Application Priority Data
Dec. 29, 2020 (TR) 2020/22155

(51) Int. Cl. A61F 2/16; A61L 31/10; A61L 31/14

(52) U.S. Cl. CPC A61F 2/1678; A61L 31/10; A61L 31/14; A61L 2430/16

(57) ABSTRACT

Crosslinked polymer coatings which provide the lubrication of the inner surface of a cartridge used as an intermediary for the implantation of intraocular lenses (IOL) in order to replace the natural lens with an artificial lens after removal of the natural lens that has lost its transparency in cataract surgery, in order to facilitate the delivery of the intraocular lens are provided. The objective of the invention is to develop a coating which will enable the intraocular lens (IOL) to be easily implanted through the cartridge without damaging it, remains stable during its long shelf life, and is flexible and lubricious.

### CROSS REFERENCE TO RELATED APPLICATIONS

This application is the national phase entry of International Application No. PCT/TR2021/051564, filed on Dec. 28, 2021, which is based upon and claims priority to Turkish Patent Application No. 2020/22155, filed on Dec. 29, 2020, the entire contents of which are incorporated herein by reference.

### FIELD OF INVENTION

The present invention relates to crosslinked polymer coatings which provide the lubrication of the inner surface of a cartridge used as an intermediary for the implantation of intraocular lenses (IOL) in order to replace the natural lens with an artificial lens after removal of the natural lens that has lost its transparency in cataract surgery, in order to facilitate the delivery of the intraocular lens.

### BACKGROUND

A cataract is the deterioration of vision due to the decrease in the light reaching the retina as a result of the natural crystal lens in the human eye losing its transparency over time due to old age, cortisone drugs, diabetes, excessive use of cigarettes and alcohol, exposure to long-term radiation or sun, impact or several diseases.

The natural crystal lens is located inside the eye, behind the iris, which is the colored part of the eye. Normally, the crystal lens focuses the light on the retina, which enables to send the image to the brain via the optic nerves. However, when the crystal lens becomes blurred due to cataract, it cannot focus properly, the light cannot be collected in a single point and it is scattered. This causes vision problems.

A cataract can be diagnosed with an eye exam. In an eye exam, examination of the eyes is done using a vision test and a slit lamp microscope. By using special eye drops, the pupil is dilated, so that the back of the eye, where the retina and optic nerve are located, can be seen better. If the concerned vision loss cannot be corrected with glasses or contact lenses, surgery may be required to remove the cataract. Cataract surgery involves removing the natural lens that has lost its transparency and replacing it with an artificial lens. The operation is usually performed as an outpatient procedure; it is very safe and effective. The most common type of cataract surgery is known as phacoemulsification (phaco). In this procedure, the doctor makes a small incision in the eye and breaks up the lens using ultrasonic waves. The natural lens is then removed and replaced with an artificial intraocular lens (IOL).

In the state of the art, these intraocular lenses (IOL) were made of polymethyl methacrylate (PMMA) material in the early days due to its biocompatibility. Since PMMA is a hard polymer, a 5-7 mm incision was required for its implantation. Since an incision of this size requires suture, it decreases the comfort of the patient and prolongs the healing process. Furthermore, PMMA must be manually foldable due to its rigid structure and it can only be inserted into the eye using forceps. Since the use of forceps can often damage the optical surface and haptics of the lens, this procedure makes it inevitable to make a wide incision using sutures. In addition, placing the IOL with manual folding requires a technical skill of use.

Recently, cataract surgery can be performed through small incisions with the discovery of innovative surgical instruments, intraocular lenses (IOLs) produced with advanced materials and functions (for example, soft and foldable materials such as silicone, hydrophobic acrylic and hydrophilic acrylic). This incision should be as small as possible to reduce trauma and accelerate recovery.

The development of an injector which can transfer the lens into the eye by opening a small incision has an important meaning for the success of minimal incision cataract surgery. Faster, more controlled and proper placement of the IOL reduces the risk of technical errors due to the implantation procedure, the risk of technical errors associated with IOL or injector damage, and post-operative complications (e.g. infection from microorganism contamination). Implantation performed with smaller incision sizes not only eliminates the need for sutures, but also accelerates the healing process of the patient. For this reason, today, acrylic based foldable, flexible intraocular lenses (IOLs) having both hydrophilic and hydrophobic properties and shape memory are produced. These lenses can be implanted into the eye even in incision sizes of 3 mm or less.

Cartridge injector systems are used for implanting the lens through a small incision into the eye in cataract surgeries. The lens is folded in the cartridge and passed through the small diameter cartridge tunnel, and then unfolded in the lens capsule located in the eye. If necessary, the surgeon can make minor interventions to bring the lens to the suitable position. When the IOL is to be placed into the eye with the injector, it is desired to pass through the tip of the injector undamaged. If an IOL is released from the injector damaged or in the wrong direction, the surgeon must remove the IOL.

IOL cartridges are generally produced from polymers such as polyolefin (e.g. polypropylene) having high hydrophobic properties. When the IOL is pushed inside the cartridge made of these polymers with high friction force, the movement of the lens inside the cartridge is prevented. As the pressure increases, IOL which is folded inside the cartridge has a tendency to expand inside the cartridge with the effect of the friction force, and it becomes possible to come out of the end of the cartridge. In this case, implantation fails and the IOL undergoes physical deformations such as rupture, tear, and scratching.

Recently, different methods have been used to minimize the friction in the cartridge and facilitate the implantation of the intraocular lens (IOL) by coming out of the cartridge tip. One of these methods is adding fatty acid esters such as glycerol monostearate (GMS) to the material of the cartridge as lubricant additive in the production process. The cartridges produced with this method are subjected to high temperatures for the lubricant additive to impregnate into the inner surface of the cartridge. Even though the cartridge added with lubricant provides a quite effective slippery coating, these fatty acid esters (lubricant) rise to the surface of the cartridge over time. This lubricant material, which has risen to the surface during its long shelf life, may adhere to the surface of the intraocular lens (IOL), causing its optical properties to be damaged. Therefore, the shelf life of the cartridges produced by this method should be kept short.

United States patent document no U.S. Pat. No. 8,323,799B2, an application known in the state of the art, discloses that a solution formed by formulating polyvinyl pyrrolidone (PVP) or hyaluronic acid (HA) as a hydrophilic lubricant, commercial urethane dispersion as a matrix polymer and polyfunctional aziridine as a crosslinking agent is used as an IOL cartridge coating material. Within the scope of the said application, it is stated that the cartridges subjected to plasma treatment are coated with the prepared coating solution and then left to dry overnight at 60° C.

Another method known in the art is to apply a polymer-based lubricant film coating on the inner surface of the cartridge. In patent applications U.S. Pat. Nos. 6,238,799B1 and 6,866,936B2 made in accordance with this method, mixtures comprising polyacrylates, polymethacrylates, polyurethanes, polyethylene and polypropylene copolymers, polyvinyl chlorides, epoxides, polyamides, polyesters and alkyd copolymers as matrix polymer; poly(N-vinyl lactams), poly(vinylpyrrolidone), poly(ethylene oxide) polypropylene oxide) polyacrylamides, cellulosic, methyl cellulose, polyacrylic acids, polyvinyl alcohols, and polyvinyl ethers as hydrophilic polymers; and at least one crosslink agent are used as IOL coating material. The coating process is performed by applying this mixture to IOL cartridges. The lubricant coatings disclosed in the said applications are relatively hard and non-flexible. This situation has a risk that the cartridge inside the cartridge may detach from the coated surface due to its hard structure and may damage the lens during the implantation process.

United States patent document no U.S. Pat. No. 8,821,572B2, an application known in the state of the art, discloses that IOL coating material comprises polyurethane and PVP which is a hydrophilic polymer and a cross-linking agent. Here, it is aimed to apply the coating directly to the inner surface of the cartridge as a single layer.

In an article named “Preparation and evaluation of a lubricious treated cartridge used for implantation of intraocular lenses”, one of the applications known in the state of the art, discloses that cartridges subjected to plasma treatment are immersed in a coating solution formed by using polyethylene imine (PEI), PVP as IOL coating material and glutaraldehyde as crosslinking agent and cured at °70 C. It is explained that after the lenses are placed in the coated cartridges, viscoelastic gel is injected before implantation to help the lens slide more easily, and it is waited for 4.5 minutes the lens to be activated. This is quite a long time and carries great risks during the surgical operation. Implantation should be performed in a short time after both viscoelastic gel and saline solution, which will ensure that the lens and coating material are activated, are added.

United States patent document no US20170128195A1, an application known in the state of the art, discloses a solution comprising polyurethane and fluorescing sodium salt as IOL cartridge coating material and polyfunctional aziridine which is a crosslinking agent. It is stated that the coated cartridges are exposed to UV light of 254 nm and the indicator properties are observed whether the fluorescent salt showing fluorescent properties is coated homogeneously on the cartridge.

### SUMMARY

The objective of the invention is to develop a coating which will enable the intraocular lens (IOL) to be easily implanted through the cartridge without damaging it, remains stable during its long shelf life, and is flexible and lubricious.

### BRIEF DESCRIPTION OF THE DRAWINGS

Crosslinked polymer coatings for intraocular lens (IOL) cartridges developed in order to fulfil the objective of the present invention is illustrated in the accompanying figures, in which:

FIG. 1—is a schematic view of the method of applying coating on the surface of intraocular lens (IOL) cartridge within the scope of the invention.

FIG. 2—is a schematic view of the crosslinking reaction within the scope of the invention.

### DETAILED DESCRIPTION OF THE INVENTION

The components shown in the FIGS. are each given reference numbers as follows:
100. method of coating the intraocular lens (IOL) cartridge surface (100)
   1. Polypropylene (PP) Cartridge
   2. Plasma Treatment
   3. PP Cartridge treated with plasma
   4. Polycation coating
   5. Polyanion coating
   6. Curing process
   7. Coated PP Cartridge

The present invention relates to crosslinked polymer coatings which provide the lubrication of the inner surface, which is particularly in direct contact with the IOL, of a cartridge used as an intermediary for the implantation of intraocular lenses (IOL) in order to replace the natural lens with an artificial lens after removal of the natural lens that has lost its transparency in cataract surgery, in order to facilitate the delivery of the intraocular lens.

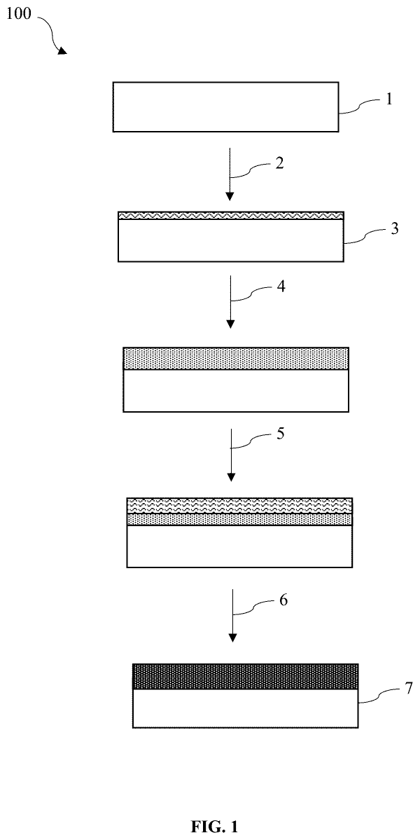

Referring to FIG. 1, the coating method (100) developed within the scope of the invention comprises the steps of
Applying plasma treatment (2) on the intraocular lens (IOL) cartridge's inner surface, which is particularly in direct contact with the IOL,
Coating the cartridge surface with the cationic functional polymer as the first layer,
Then, applying coating on the cationic functional polymer coating on the cartridge surface as a second layer with the solution comprising anionic functional polymer and crosslinker,
Performing the curing process (6) on the coating surfaces.

Acrijet Fly polypropylene (PP) cartridges (1), produced by VSY Biotechnology, suitable for 2.0 mm micro-incision cataract surgery were subjected to plasma treatment (2) and thereby their surfaces were made hydrophilic. The plasma treatment (2) was carried out for 3 minutes with a gas flow of 0.30 mbar at 100 W power.

The coatings for the intraocular lens (IOL) cartridge developed within the scope of the present invention are crosslinked polymeric coating materials which are used as a coating on the inner surfaces of the intraocular lens cartridges, and which facilitate the implantation of intraocular lenses. The subject matter of the invention enables to develop a coating, which will enable the intraocular lens (IOL) to be easily implanted through the cartridge without damaging it, remains stable during its long shelf life, and is flexible and lubricious.

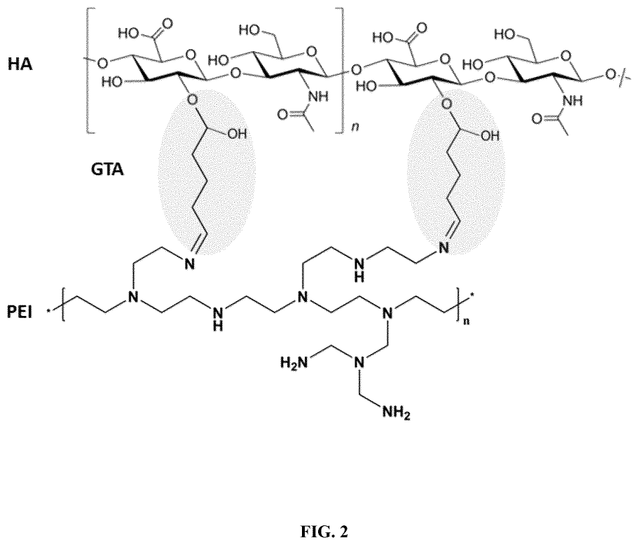

Within the scope of the invention, two different polymers are used in terms of functional properties. As the first layer, amine functional cationic polyethyleneimine (PEI) is used; and as the second layer carboxylic acid and hydroxyl functional anionic polymer hyaluronic acid (HA) are used. Glutaraldehyde (GTA) is used as a crosslinking agent to ensure crosslinking of the polymers among each other. The interaction between the polymers and the crosslinker is shown in FIG. 2.

Dip coating method is preferably used as the coating method within the scope of the invention. PP cartridges (1) subjected to the plasma treatment (2) were immersed in PEI solutions for 10 seconds prior to being immersed in the formulations prepared in deionized water, the weight ratios of which are given in Table 1 (in proportion to the total weight of the coating solutions). Cartridges preferably coated with 2.5-10% by weight of PEI were then dried at 80° C. for 30 min. The dried cartridges were then immersed in HA+GTA solutions (preferably 0.25-1% HA and 1% GTA by weight) among the formulations mentioned in Table 1 for 10 seconds and left for the crosslinking reaction (curing process (6)) at 80° C. for 60 minutes, thereby ensuring the adhesion of the film to the cartridge surface. The GTA ratio was kept constant at 1% by weight in all formulations. After the crosslinking reaction, the PP cartridges (1) were immersed in the ethanol solution so that the impurities remaining or formed in the medium after the reaction were removed; after the removal process the PP cartridges (1) were dried at 60° C. for 30 minutes.

TABLE 1
Compositions by weight of the
solutions used in the coating trials

| Trial | 1^{st} Layer | 2^{nd} Layer | |
|---|---|---|---|
| 1 | 2.5% PEI | 0.25% | HA |
| | | 1.00% | GTA |
| 2 | 2.5% PEI | 0.50% | HA |
| | | 1.00% | GTA |
| 3 | 2.5% PEI | 1.00% | HA |
| | | 1.00% | GTA |
| 4 | 5.00% PEI | 0.25% | HA |
| | | 1.00% | GTA |
| 5 | 5.00% PEI | 0.50% | HA |
| | | 1.00% | GTA |
| 6 | 5.00% PEI | 1.00% | HA |
| | | 1.00% | GTA |
| 7 | 10.00% PEI | 0.25% | HA |
| | | 1.00% | GTA |
| 8 | 10.00% PEI | 0.50% | HA |
| | | 1.00% | GTA |
| 9 | 10.00% PEI | 1.00% | HA |
| | | 1.00% | GTA |

The expression “1^{st} layer” indicates the composition of the solution belonging to the first layer in the coating process of the cartridges after the plasma treatment (2). For example, the first layer used in Trial 1 comprises 2.5% PEI relative to the weight ratio of the solution in which it was prepared. After the first coating made with this coating solution, the drying process is carried out and the cartridge becomes coated with the first layer. The expression “2nd layer” indicates the composition of the solution belonging to the second layer of the cartridge coated with the first layer in the coating process. For example, the second layer used in Trial 1 comprises 0.25% HA and 1.00% GTA relative to the weight ratio of the solution in which it was prepared. After the cartridge is coated with the second layer, the curing process (6) is performed and then the impurities are removed in ethanol. As a final step, the cartridges are dried and made ready for use.

Delivery tests of PP cartridges (1) coated with polymer solutions at different ratios, the adhesion properties of the coating to the surface during delivery (whether the coating comes off from the tip of the cartridge with the lens) and the injection force tests were examined.

### Experimental Studies

### Delivery Tests

Within the scope of the experimental studies carried out while developing the invention, the delivery tests of hydrophilic acrylic intraocular lenses (IOL) with the cartridges obtained as a result of the curing process (6) were performed. The evaluation of the delivery tests was carried out using the same type of intraocular lenses (Acriva UD 613, VSY Biotechnology) with middle diopter power. The delivery tests were performed with the following process steps:
1. Squeezing viscoelastic gel (Protectalon 1.4%, VSY Biotechnology) into the coated cartridge
2. Placing the lens in the cartridge
3. Mounting the cartridge into the injector
4. Performing the delivery tests under optic microscope
5. Checking the optical/haptic parts of the lens and checking whether the coating comes off with the lens

The obtained results are summarized in Table 2.

TABLE 2
Evaluation of Delivery Tests

| Trial | Delivery test | Observations | Coating Transfer |
|---|---|---|---|
| 1 | ++ | Haptic damage | Not observed |
| 2 | ++ | Haptic damage | Not observed |
| 3 | ++ | Haptic damage | Not observed |
| 4 | +++ | Smooth deliveries, | Not observed |
| | | no optic/haptic | |
| | | damage | |
| 5 | +++ | Smooth deliveries, | Not observed |
| | | no optic/haptic | |
| | | damage | |
| 6 | +++ | Smooth deliveries, | Not observed |
| | | no optic/haptic | |
| | | damage | |
| 7 | +++ | Smooth deliveries, | Not observed |
| | | no optic/haptic | |
| | | damage | |
| 8 | +++ | Smooth deliveries, | Not observed |
| | | no optic/haptic | |
| | | damage | |
| 9 | +++ | Smooth deliveries, | Not observed |
| | | no optic/haptic | |
| | | damage | |

+++ Passed
++ Passed with difficulty
+ Did not pass

According to the results shown in Table 2, no coating transfer was observed during the delivery from the cartridge tip in any of the delivery trials, and deliveries were successful, except Trials 1, 2 and 3. In Trials 1, 2 and 3, damages occurred in the haptics of the lenses due to the strain during the delivery. In the other trials, the deliveries were performed smoothly, and no deformation was observed in the haptics or optics of the lens. This problem in Trials 1, 2 and 3 can be considered to be due to the fact that the coating is not lubricious enough, that is, the amount of polymer compositions in the coating solution is low. The importance of these polymer compositions, which reduce friction in the cartridge, is clearly seen in the table.

### Injection Force Tests

One of the most important factors during the implantation of IOLs into the eye is the injection force to be applied during the delivery of the lens through the cartridge injector system. Injection force is generally desired to be below 35 N. This is because when high injection force is applied, it becomes difficult to insert the lens into the eye and damages (scratches on the lens, haptic and optic damages) occur in the IOL. In addition, the cartridge injector system may also get damaged (cartridge cracking).

Injection force tests required to be applied during the implantation of Acriva UD 613 IOLs were performed at 200 mm/min using the Lloyd-LS1 test system. In all tests, the middle diopter Acriva UD 613 IOL were used, and Protectalon 1.4% viscoelastic gel was added into cartridges to allow the lens to interact with the coating material and move forward. The results are summarized in Table 3.

TABLE 3
Results of injection force applied during delivery tests

| Trial | Injection Force (N) |
|---|---|
| 1 | 46.7 |
| 2 | 40.3 |
| 3 | 36.5 |
| 4 | 17.4 |
| 5 | 17.9 |
| 6 | 15.8 |
| 7 | 13.4 |
| 8 | 11.8 |
| 9 | 12.6 |

In the results of the injection force shown in Table 3, the force applied in trials 1, 2 and 3, in which the lens passed through the cartridge with difficulty, exhibited a value above 35 N. In these trials, even though the lens could pass through the cartridge, though with difficulty, it damaged the haptic parts of the lens. This is not suitable for implantation. In other trials (trials 4-9), where the delivery tests were flawless, the injection force results were lower than 35 N and exhibited proper values for flawless implantation. The results of these trials, which showed values well below 35 N, indicate that the coatings performed are suitable for IOL cartridges.

What is claimed is:

1. A cartridge for facilitating delivery of an artificial intraocular lens (IOL) for implantation after removal of the natural lens, comprising:
a crosslinked polymer coating which provide lubrication of the inner surface of the cartridge, and which is in direct contact with the IOL.

2. A method of obtaining the cartridge having an inner surface provided with crosslinked polymer coatings disclosed in claim 1, comprising the steps of:
applying plasma treatment on the intraocular lens (IOL) cartridge inner surface, which is in direct contact with the IOL,
coating the cartridge surface with the cationic functional polymer as the first layer,
then, coating the cationic functional polymer coating on the cartridge surface with a second layer solution comprising anionic functional polymer and crosslinker,
performing a curing process on the coating surfaces.

3. The method of obtaining the cartridge having the inner surface provided with crosslinked polymer coatings according to claim 2, wherein the cartridge is polypropylene and the inner surfaces of the polypropylene cartridge is subjected to plasma treatment carried out for 3 minutes with a gas flow of 0.30 mbar at 100 W power in order to make them hydrophilic.

4. The method of obtaining the cartridge having the inner surface provided with crosslinked polymer coatings according to claim 2, wherein amine functional cationic polyethyleneimine polymer is used as the first layer.

5. The method of obtaining the cartridge having the inner surface provided with crosslinked polymer coatings according to claim 4, wherein 2.5-10% by weight of polyethyleneimine polymer is used.

6. The method of obtaining the cartridge having the inner surface provided with crosslinked polymer coatings according to claim 2, wherein carboxylic acid and hydroxyl functional anionic hyaluronic acid polymer are used as the second layer.

7. The method of obtaining the cartridge having the inner surface provided with crosslinked polymer coatings according to claim 6, wherein 0.25-10% by weight of hyaluronic acid polymer is used.

8. The method of obtaining the cartridge having the inner surface provided with crosslinked polymer coatings according to claim 2, wherein glutaraldehyde is used as the crosslinker to ensure crosslinking of polymers.

9. The method of obtaining the cartridge having the inner surface provided with crosslinked polymer coatings according to claim 8, wherein 1-10% by weight of glutaraldehyde is used.

10. The method of obtaining the cartridge having the inner surface provided with crosslinked polymer coatings according to claim 2, wherein the cartridges are polypropylene and are subjected to the plasma treatment and then are immersed in polyethyleneimine as the cationic functional polymer solution for 10 seconds prior to being immersed in the solution comprising anionic functional polymer and crosslinker that is prepared in deionized water.

11. The method of obtaining the cartridge having the inner surface provided with crosslinked polymer coatings according to claim 2, wherein the cartridge coated with polyethyleneimine as the cationic functional polymer is then dried at 80° C. for 30 min.

12. The method of obtaining the cartridge having the inner surface provided with crosslinked polymer coatings according to claim 2, wherein cartridge coated with polyethyleneimine as the cationic functional polymer is then immersed in the solution comprising anionic functional polymer and crosslinker, and which comprises hyaluronic acid and glutaraldehyde for 10 seconds.

13. The method of obtaining the cartridge having the inner surface provided with crosslinked polymer coatings according to claim 2, wherein the cartridge immersed in the solution comprising anionic functional polymer and crosslinker, and which comprises hyaluronic acid and glutaraldehyde are left for the curing process which is a crosslinking reaction at 80° C. for 60 minutes.

14. The method of obtaining the cartridge having the inner surface provided with crosslinked polymer coatings according to claim 2, wherein, after the curing process, the polypropylene cartridges are immersed in ethanol solution so that the impurities remaining or formed in the medium after the reaction are removed, and then the polypropylene cartridges are dried at 60° C. for 30 minutes.

15. The method of obtaining the cartridge having the inner surface provided with crosslinked polymer coatings according to claim 3, wherein glutaraldehyde is used as the crosslinker to ensure crosslinking of polymers.

16. The method of obtaining the cartridge having the inner surface provided with crosslinked polymer coatings according to claim 4, wherein glutaraldehyde is used as the crosslinker to ensure crosslinking of polymers.

17. The method of obtaining the cartridge having the inner surface provided with crosslinked polymer coatings according to claim 5, wherein glutaraldehyde is used as the crosslinker to ensure crosslinking of polymers.

18. The method of obtaining the cartridge having the inner surface provided with crosslinked polymer coatings according to claim 6, wherein glutaraldehyde is used as the crosslinker to ensure crosslinking of polymers.

19. The method of obtaining the cartridge having the inner surface provided with crosslinked polymer coatings according to claim 7, wherein glutaraldehyde is used as the crosslinker to ensure crosslinking of polymers.

20. The cartridge of claim 1, wherein the crosslinked polymer coating comprises:
a first layer formed from polyethyleneimine disposed on the inner surface of the cartridge, and
a second layer comprising hyaluronic acid and a crosslinker comprising glutaraldehyde disposed on the first layer.
